(19) United States
(12) Patent Application Publication
(10) Pub. No.: US 2025/0339240 A1
(43) Pub. Date: Nov. 6, 2025

(54) OPTIMIZED MYOFUNCTIONAL DEVICE

(71) Applicant: Maria Amoruso, Bari (IT)

(72) Inventor: Maria Amoruso, Bari (IT)

(21) Appl. No.: 18/855,472

(22) PCT Filed: Apr. 5, 2023
(86) PCT No.: PCT/IB2023/053484

§ 371 Date: Oct. 9, 2024

(30) Foreign Application Priority Data
Apr. 12, 2022 (IT) 102022000007223

(51) Int. Cl. A61C 7/08

(52) U.S. Cl. CPC A61C 7/08

(57) ABSTRACT

The present invention relates to a myofunctional device, optimized to realize a treatment apt to obtain a correct dental occlusion and a correct lip seal.

It is a myofunctional device which can be used together with teeth aligners, and which can be optimized to maximize the comfort of usage by the user and the efficacy of treatment at the same time.

### BACKGROUND OF THE INVENTION

### 1. Field of the Invention

The present invention relates to a myofunctional device, optimized to realize a treatment apt to obtain a correct dental occlusion and a correct lip seal more efficiently than what occurs with the devices known at the state of the art.

### 2. Brief Description of the Prior Art

Myofunctional orthodontics consists in the reeducation of the lips, cheeks and tongue muscles, as well as in the orthodontic correction by using specific devices.

At the state of the art, there are known many examples of myofunctional devices, realized to treat and solve class II, class Ill dental crowding. Such devices are also used in children as interceptive orthodontics to guide and correct the bad growth of teeth and maxillary bones, and in adults for the treatment of symptoms as migraine, dizziness, tinnitus, malocclusion, bad posture.

### Technical Problem Solved

None of the devices known at the state of the art allows the use with teeth aligners; moreover, none of the devices known at the state of the art is optimized to maximize the comfort of usage by users and the efficacy of treatment.

Moreover, none of the known devices is optimized to maximize the efficacy of lip seal correction, or to allow an effective outflow of saliva without needing to remove the device from the oral cavity.

### Aim of the Invention

Therefore, aim of the present invention is to provide a myofunctional device which can be used together with teeth aligners, and which can be optimized to maximize the comfort of usage by the user and the efficacy of treatment at the same time.

### SUMMARY OF THE INVENTION

The device realizes the prefixed aims since it is a myofunctional device (1) comprising an inner arch (12) and an outer arch (13) connected to each other by an occlusal plane (14), said arches (12, 13) and said plane (14) being configured to be introduced in the oral cavity of a patient, so that: the occlusal plane (14) is arranged between the upper dental arch and the lower one; the inner arch (12) is arranged inside the two dental arches, the outer arch (13) is arranged outside the two dental arches, in the space comprised between the same and the inner mucosa of cheeks and lips, characterized in that in the front portion of the outer arch (13) the device (1) comprises a plurality of horizontal reliefs (131), arranged at opposite portions to the middle horizontal plane of the device, in a number of at least two for any portion.

### BRIEF DESCRIPTION OF THE DRAWINGS

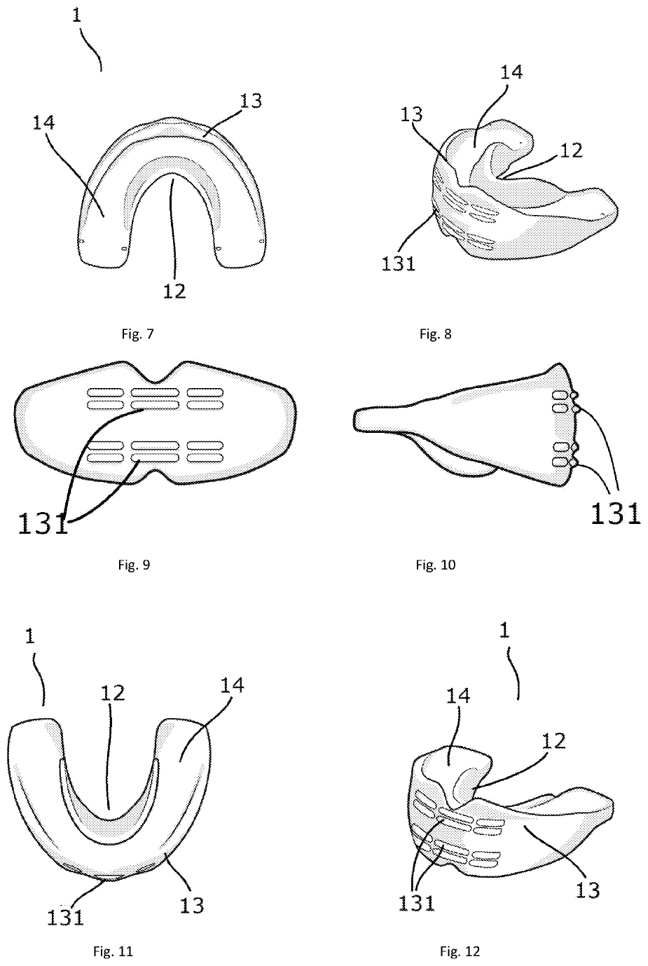

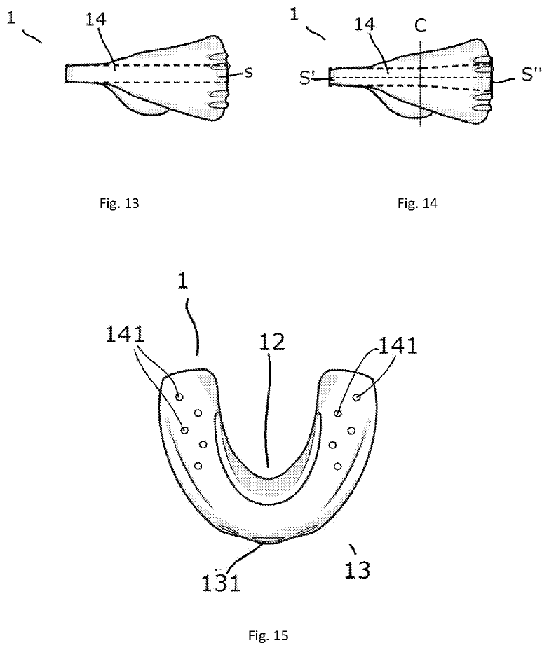

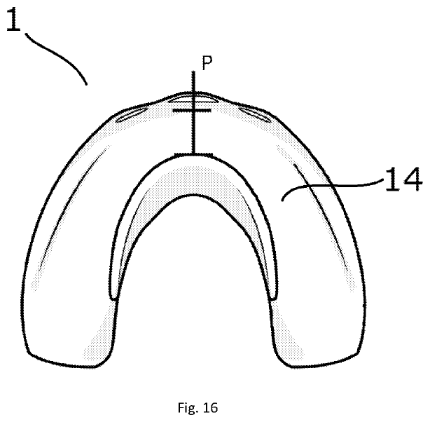

The appended FIGS. 1 to 6 show views from various angles of a first embodiment of the device according to the invention; FIGS. 7 to 12 show views of a second embodiment; FIGS. 13 and 14 show two illustrative views of the thickness variation of the occlusal plane; FIG. 15 shows an illustrative view of a possible arrangement of the holes for the saliva outflow.

### DETAILED DESCRIPTION OF THE INVENTION

With reference to the appended figures, the invention provides a myofunctional device (1) comprising an inner arch (12) and an outer arch (13) connected to each other by an occlusal plane (14), said arches (12, 13) and said plane (14) being configured to be introduced in the oral cavity of a patient, so that:
the occlusal plane (14) is arranged between the upper dental arch of the patient and the lower one,
the inner arch (12) is arranged inside the two dental arches of the patient,
the outer arch is arranged outside the two dental arches of the patient, in the space comprised between the same and the inner mucosa of cheeks and lips.

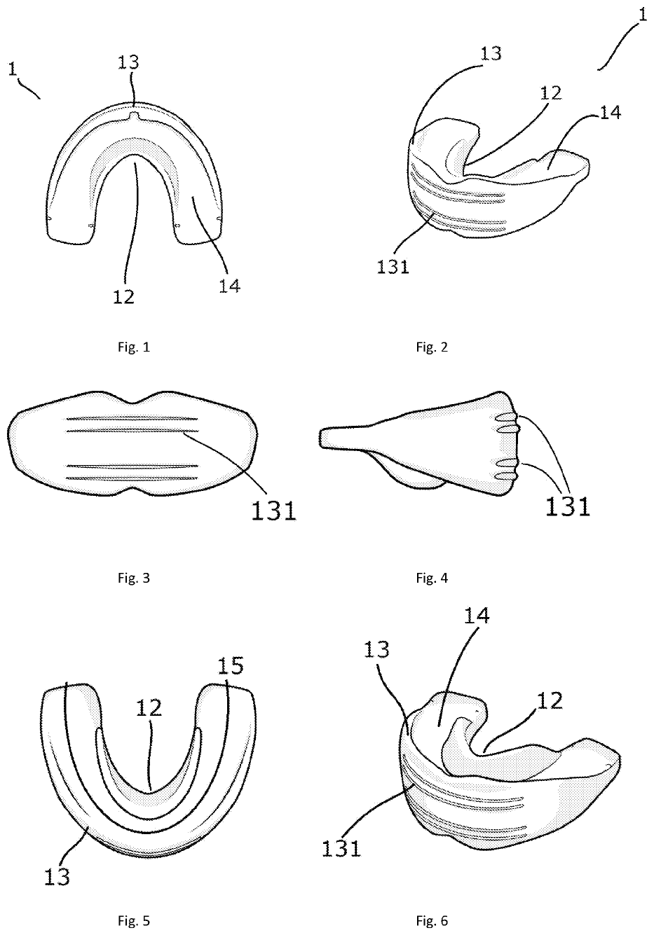

It is to be specified that with inner arch (12) and outer arch (13), as it is shown for example in FIG. 2, it is intended portions of the device with greater thickness than the occlusal plane (14), positioned respectively inside and outside the same and projecting both above and under the same.

As it is shown in the appended figures, in the front portion of the outer arch (13) the device (1) comprises a plurality of horizontal reliefs (131), arranged at opposite portions to the occlusal (middle) plane of the device, in a number of at least two for any portion.

To such aim it is to be specified that the middle horizontal plane of the device is defined by the occlusal plane (14).

In a first embodiment, such reliefs (131) are shaped as continuous segments; in a second embodiment such reliefs are shaped as discontinuous segments.

The function of the just described reliefs (131) is to allow the patient, both child and adult, to obtain, in a more efficient and natural way, a more precise lip seal than what occurs with the devices known at the state of the art, which are not provided with any relief, or which have round shaped front reliefs or similar.

It is to be specified that with lip seal it is intended the contact between upper lip and lower lip, at rest and without muscle effort. This allows a better physiological deglutition, in addition to a better nasal breathing and a more fluid and comprehensible phonation.

Another feature of the device according to the invention is that the depth (P) of the occlusal plane (14), intended as distance between the inner arch (12) and outer arch (13) is such that it allows the use of the device also by subjects using teeth aligners.

These are devices known at the state of the art with many trade names (ex. Invisalign) which consist in transparent masks worn on teeth with corrective action.

To such aim, the depth of the occlusal plane (14), measured inside the lifted edges defining the outer arch and the inner arch and at the middle of the device, as it is shown in FIG. 16, is at least 5.5 mm, and preferably greater than 6.5 mm. The shape of the device is also such that the middle is the point where the depth of the occlusal plane (14) is the lowest.

The technical effect of this variation is double. On the one hand, it allows the user of teeth aligners to be able to use also the myofunctional device according to the invention, thus obtaining all the advantages in terms of malocclusion treatment, correction of lip seal, posture etc.

On the other hand, the treatment with teeth aligners becomes more effective and faster. In fact, the presence of the myofunctional device, and the muscle contractions as well as the consequent gingival and dental stimulations increase the blood supply to the periodontal fiber.

Moreover, the presence of the myofunctional device increases also the adherence of the aligners (invisible masks) to teeth, forcing constantly all the dental elements to follow the movements given by the aligners.

Another feature of the device according to the present invention is that preferably the thickness of the occlusal plane (14), measured along the development of the middle arch (15), is not constant.

In a first embodiment, the thickness of the occlusal plane (14) at the front portion of the device is greater than the thickness in the two side portions. In a second embodiment, the thickness of the occlusal plane (14) at the front portion of the device is lower than the thickness in the two side portions.

In a subject without any problems of hypo-or hyper-divergence the occlusal plane (14) of the device has a constant thickness(S), preferably between 4 and 6 mm, and indicatively equal to 5 mm.

In a subject with hyper-divergence problems the occlusal plane (14) has a constant thickness(S), lower than the one suitable for subjects without problems, preferably between 2 and 3 mm, and indicatively equal to 2.5 mm.

In a subject with hypo-divergence problems, the occlusal plane has a variable thickness, greater in the front portion of the device than in the end portion. In particular, according to a preferred embodiment, the occlusal plane (14) has a constant thickness (S′) in the portion at the rear of the position (C) of the upper canine tooth, and an increasing thickness from the canine position to the front portion of the device. Preferably, the constant thickness of the portion at the rear of the position of the upper canine tooth is between 2 and 3 mm, and indicatively is equal to 2.5 mm, and the increase in thickness in the front portion is such that the whole maximum thickness (S″) is about 4 mm in the front portion of the device.

Obviously little variations in the dimensions indicated are possible, without departing from the aims of the invention.

Preferably, but not limitingly, the device (1) comprises also a plurality of through holes (141) along the whole development of the occlusal plane (14).

Yet, more preferably, the device comprises a plurality of through holes in the outer portion of the device, having axis inclined to the horizontal, so to allow the saliva to flow therethrough, and so to be swallowed as it occurs physiologically. This makes the use of the device more comfortable, and the patient does not feel the need to remove the device from the mouth to remove and clean the saliva excess formed on the upper and lower plane of the device.

1. A myofunctional device (1) comprising an inner arch (12) and an outer arch (13) connected to each other by an occlusal plane (14), said arches (12, 13) and said plane (14) being configured to be introduced in the oral cavity of a patient, so that:
the occlusal plane (14) is arranged between the upper dental arch of the patient and the lower one;
the inner arch (12) is arranged inside the two dental arches of the patient,
the outer arch (13) is arranged outside the two dental arches, in the space comprised between the same and the inner mucosa of cheeks and lips,
characterized in that
in the front portion of the outer arch (13) the device (1) comprises a plurality of horizontal reliefs (131), arranged at opposite portions to the middle horizontal plane of the device, which is defined by said occlusal plane (14), in a number of at least two for any portion.

2. The device according to claim 1, wherein said reliefs (131) are shaped as continuous segments.

3. The device according to claim 1, wherein said reliefs (131) are shaped as discontinuous segments.

4. The device according to claim 1, wherein the depth (P) of said occlusal plane (14), intended as distance between the inner arch (12) and outer arch (13) is such that it allows the use of the device also by subjects using teeth aligners.

5. The device according to claim 4, wherein said depth (P) of said occlusal plane (14), measured inside said inner arch (12) and said outer arch (13) at the middle of the device, is greater than 5.5 mm, and in that the shape of the device is also such that the middle is the point where the depth of the occlusal plane (14) is the lowest one.

6. The device according to claim 1, wherein the thickness(S) of said occlusal plane (14), measured along the development of the middle arch (15), is constant.

7. The device according to claim 6, wherein said thickness(S) is between 4 and 6 mm.

8. The device according to claim 1, wherein the thickness of said occlusal plane (14) measured along the development of the middle arch (15) is greater in the front portion of the device than in the end portion.

9. The device according to claim 8, wherein said occlusal plane (14) has a constant thickness (S′) in the portion at the rear of the position (C) of the upper canine tooth, and an increasing thickness from the canine position to the front portion of the device.

10. The device according to claim 1, further comprising a plurality of through holes (141) arranged along the development of said occlusal plane (14).
